(19) United States
(12) Patent Application Publication
(10) Pub. No.: US 2015/0093749 A1
(43) Pub. Date: Apr. 2, 2015

(54) REAL-TIME PCR DETECTION OF STREPTOCOCCUS PYOGENES

(71) Applicant: VELA OPERATIONS PTE. LTD., Singapore (SG)

(72) Inventor: Ann Lee Ling, Singapore (SG)

(21) Appl. No.: 14/381,415

(22) PCT Filed: Feb. 28, 2013
(86) PCT No.: PCT/IB2013/051590

§ 371 Date: Aug. 27, 2014

(30) Foreign Application Priority Data
Feb. 29, 2012 (GB) 1203568.9

(51) Int. Cl. C12Q 1/68

(52) U.S. Cl. CPC C12Q 1/689; C12Q 2600/158
USPC 435/6.11

(57) ABSTRACT

The present invention relates to assays, diagnostic kits and methods for real-time PCR detection of S. pyogenes.

### TECHNICAL FIELD OF THE INVENTION

The present invention relates to diagnostic assays for the detection of Streptococcus pyogenes using real-time PCR.

### BACKGROUND

Streptococcus pyogenes (S. pyogenes) belongs to gram-positive bacteria causing Group A streptococcal infections. S. pyogenes is responsible for a large number of infections in humans, ranging from skin infections to systemic diseases which may be life-threatening and in particular pharyngitis, which may be associated with the release of certain toxins leading to scarlet fever. Other diseases associated with S. pyogenes are sinusitis, lymphadenitis, pyoderma, endocarditis, meningitis, septicaemia, tonsillitis, impetigo. It is also assumed that S. pyogenes infections may be responsible for certain autoimmune diseases, e.g. rheumatic fever. S. pyogenes is sensitive to penicillin, although certain strains have developed resistance to macrolytes, tetracycline and a few other antibiotics.

Currently, the specific diagnosis of S. pyogenes is performed on material obtained by throat swab, culturing on blood agar and various other time-consuming and expensive tests such as antibody-based tests, etc.

In the recent past, biochemical methods have been developed to detect S. pyogenes, but the sensitivity and time necessary for detection of S. pyogenes nucleic acids, e.g. DNA or RNA, is not always satisfactory. In particular, small amounts of nucleic acids in the range of 10 to 100 femtogram are extremely difficult.

Accordingly, a highly sensitive and rapid qualitative real-time PCR assay for the detection and diagnosis of an infection with S. pyogenes based on conserved regions of the S. pyogenes exotoxin B (SpeB) gene of all available S. pyogenes strains is provided.

### SUMMARY OF THE INVENTION

The present invention relates to a set of nucleic acids, useful for detection of S. pyogenes in a biological sample, the set comprising at least one pair of primers and a probe, specific for S. pygenes.

In a first embodiment, the first set of primers comprises sequences selected from SEQ ID Nos: 1 and 2 or SEQ ID Nos: 4 and 5, or complements thereof, or primers having at least 80%, 90%, 95%, 96%, 97%, 98% or preferably 99% homology to any of SEQ ID Nos: 1 and 2 or SEQ ID Nos: 4 and 5.

In another embodiment, the primers consist of the sequences depicted in SEQ ID Nos: 1 and 2 or SEQ ID Nos: 4 and 5, or complements or homologs thereof, and the probe has the sequence of SEQ ID No. 3, or is a complement or homolog thereof having at least 80%, 90%, 95%, 96%, 97%, 98% or preferably 99% homology.

The present invention also relates to a method for the detection of S. pygenes comprising:
a) providing a biological sample from a patient;
b) extracting nucleic acids (RNA or DNA) from the biological sample;
c) carrying out a (RT-)PCR with the set of primers according to the present invention; and
d) detecting amplification products for S. pygenes using the herein described probes, wherein the presence of an amplification product is indicative of the presence of the bacerium in the biological sample.

In an alternative method, instead of carrying a RT-PCR, the method comprises a step of reverse-transcription and a step of PCR amplification.

The present invention further concerns the use of a set of nucleic acids according to the present invention for detecting S. pyogenes. It concerns also a method of detecting S. pyogenes by using a set of nucleic acids according to the present invention. In addition, it concerns a set of nucleic acids according to the present invention for preparing a diagnostic kit useful for detecting S. pyogenes. Optionally, the kit further comprises other components such as a DNA polymerase, a reverse-transcriptase, RNase inhibitors, dNTPs and a PCR and/or RT-buffers.

### SHORT SUMMARY OF PREFERRED EMBODIMENTS

Some of the preferred embodiments of the invention are depicted below:
i. A method for the detection of the presence or absence of at least one nucleic acid specific for S. pyogenes in a biological sample, wherein the method comprises conducting real-time PCR.
ii. The method according to (i) further comprising isolating (extracting) nucleic acids from the biological sample and/or performing a reverse transcription step.
iii. The method according to any one of (i) or (ii), wherein at least one primer set that is specific for at least on S. pyogenes-specific nucleic acid is used.
iv. The method according to any one of (i) to (iii), wherein the S. pyogenes-specific primer set comprises oligonucleotide sequences set forth in SEQ ID Nos: 1 and 2, or complements or homologues thereof.
v. The method according to any one of (i) to (iii), wherein the S. pyogenes primer set consists of oligonucleotide sequences set forth in SEQ ID No: 1 and SEQ ID No: 2 or SEQ ID Nos: 4 and 5, or complements or homologues thereof
vi. The method according to any one of (i) to (v), wherein at least one probe specifically binding to a nucleic acid of S. pyogenes, preferably a probe specifically binding to a nucleic acid of S. pyogenes is used that has been amplified using the primers set forth in the preceding embodiments.
vii. The method according to any one of (i) to (vi), wherein the at least one probe comprises the sequence set forth in SEQ ID No: 3, or complements or homologues thereof.
viii. The method according to any one of (i) to (vii), wherein the primers and/or probes carry a fluorescent moiety.
ix. A method for the diagnosis of an S. pyogenes infection comprising performing one of the methods according to any one of the preceding embodiments.
x. A method of monitoring the treatment of S. pyogenes infection, said method comprising performing the method according to embodiment (ix) before treatment with at least one anti-bacterial drug and during and/or after treatment with said anti-bacterial drug, wherein the method according to embodiment (ix) that is used after treatment with the at least on anti-bacterial drug may be used at least about 24 hours, at least about 48 hours, at least about 72 hours, at least about 4 or 5 days after starting the treatment, or after subsequent to the end of the treatment period.
xi. An assay for detection of at least nucleic acid of S. pyogenes in a biological sample comprising primers specifically hybridizing to nucleic acids derived from said S. pyogenes, wherein said assay is suitable for real-time PCR.
xii. The assay according to (xi), wherein the assay comprises primers and/or probes set forth in any one of embodiments (iv) to (viii).
xiii. The assay according to embodiment (xi) or (xii), wherein the assay is adapted for use in a fully automated laboratory.
xiv. A diagnostic composition comprising primers and/or probes set forth in any one of embodiments (iv) to (viii).
xv. A diagnostic kit for the detection of S. pyogenes in a biological sample comprising primers and/or probes set forth in any one of embodiments (iv) to (viii), optionally comprising instructions for use.
xvi. The diagnostic kit according to embodiment (xv), wherein said kit further comprises enzymes, deoxynucleotides and/or buffer for a reverse transcription step and/or a PCR step.
xvii. The diagnostic kit according to any one of embodiments (xv) or (xvi) further comprising reagents for the isolation of nucleic acids from a biological sample.

### SUMMARY

The invention provides for methods of identifying S. pyogenes DNA or RNA by real-time polymerase chain reaction (PCR) in a biological sample. Primers and probes for detecting S. pyogenes are also provided by the invention, as are kits or compositions containing such primers and probes.

Methods of the invention can be used to identify RNA from specimens for diagnosis of S. pyogenes infection. The specific primers and probes of the invention that are used in these methods allow for the amplification and monitoring the development of specific amplification products.

According to one aspect of the invention, a method for detecting the presence or absence of S. pyogenes in a biological sample from an individual is provided. The method may comprise a reverse transcription step, at least one cycling step, which includes an amplifying step and a hybridizing step. The amplifying step includes contacting the sample with at least one pair of specific primers to produce an amplification product if an S. pyogenes-specific nucleic acid molecule is present in the sample. The hybridization step includes contacting the sample with S. pyogenes-specific probes. In the assays of the present invention primer pairs are used that are suitable to hybridize to nucleic acids of all known S. pyogenes strains, but not to other nucleic acids of other species. As a result of the methods described herein, the amplification and subsequent detection of the target bacteria is possible. A pair of S. pyogenes primers comprises a first S. pyogenes primer and a second S. pyogenes primer. Representative sequences of primers and the probes of the invention are shown in the sequence listing.

In some aspects of the invention, the primers and/or probes of the invention can be labeled with a fluorescent moiety. Fluorescent moieties for use in real-time PCR detection are known to persons skilled in the art and are available from various commercial sources, e.g. from life Technologies™ or other suppliers of ingredients for real-time PCR.

Representative biological samples from the respiratory tract include wound and throat swabs, throat washings, nasal swabs, and specimens from the lower respiratory tract.

In addition, the cycling step can be performed on a control sample. A control sample can include the same portion of the S. pyogenes nucleic acid molecule. Alternatively, a control sample can include a nucleic acid molecule other than an S. pyogenes nucleic acid molecule.

Cycling steps can be performed on such a control sample using a pair of control primers and a pair of control probes. The control primers and probes are different from S. pyogenes primers and probes.

One or more amplifying steps produces a control amplification product. Each of the control probes hybridizes to the control amplification product.

In another aspect of the invention, there are provided articles of manufacture, or kits.

Kits of the invention can include at least one pair of specific primers for the amplification of S. pyogenes and at least one S. pyogenes probe hybridizing specifically with the amplification products.

Articles of manufacture can include fluorophoric moieties for labeling the primers or probes or the primers and probes are already labeled with donor and corresponding acceptor fluorescent moieties.

The article of manufacture can also include a package insert having instructions thereon for using the primers, probes, and fluorophoric moieties to detect the presence or absence of S. pyogenes in a sample.

In another aspect of the invention, there is provided a method for detecting the presence or absence of S. pyogenes in a biological sample from an individual. Such a method includes performing at least one cycling step. A cycling step include at least one amplifying step and a hybridizing step. Generally, an amplifying step includes contacting the sample with a pair of primers to produce an amplification product if an S. pyogenes nucleic acid molecule is present in the sample. Generally, a hybridizing step includes contacting the sample with an S. pyogenes-specific probe. The probe is usually labeled with at least one fluorescent moiety. The presence or absence of fluorescence is indicative of the presence or absence of S. pyogenes in said sample.

Amplification generally involve the use of a polymerase enzyme. Suitable enzymes are known in the art, e.g. Taq Polymerase, etc.

In another aspect of the invention, there is provided a method for detecting the presence or absence of S. pyogenes in a biological sample from an individual. Such a method includes performing at least one cycling step. A cycling step can include an amplifying step and a dye-binding step. An amplifying step generally includes contacting the sample with a pair of S. pyogenes-specific primers to produce an S. pyogenes amplification product if an S. pyogenes nucleic acid molecule is present in the sample. A dye-binding step generally includes contacting the S. pyogenes amplification product with a double-stranded DNA binding dye. The method further includes detecting the presence or absence of binding of the double-stranded DNA binding dye into the amplification product. According to the invention, the presence of binding is typically indicative of the presence of S. pyogenes nucleic acid in the sample, and the absence of binding is typically indicative of the absence of S. pyogenes nucleic acid in the sample. Such a method can further include the steps of determining the melting temperature between the amplification product and the double-stranded DNA binding dye. Generally, the melting temperature confirms the presence or absence of S. pyogenes nucleic acid. Representative double-stranded DNA binding dyes include SYBRGREEN I®, SYBRGOLD®, and ethidium bromide.

In another aspect, the invention allows for the use of the methods described herein to determine whether or not an individual is in need of treatment for S. pyogenes.

Treatment for S. pyogenes can include, e.g., administration of antibiotics, e.g. tetracyclines or macrolides to the individual.

The invention also provides for the use of the articles of manufacture described herein to determine whether or not an individual is in need of treatment for S. pyogenes.

Further, the methods and/or the articles of manufacture described herein can be used to monitor an individual for the effectiveness of a treatment for S. pyogenes as well as in epidemiology to monitor the transmission and progression of S. pyogenes from individuals to individuals in a population. The methods and/or the articles of manufacture (e.g., kits) disclosed herein can be used to determine whether or not a patient is in need of treatment for S. pyogenes.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will be decisive.

The details of one or more embodiments of the invention are set forth in the description below. Other features, objects, and advantages of the invention will be apparent from the description, and from the claims.

### DETAILED DESCRIPTION

According to the present invention, a real-time PCR assay for detecting S. pyogenes nucleic acid in a biological sample that is more sensitive and specific than existing assays is described herein.

Primers and probes for detecting S. pyogenes infections and articles of manufacture containing such primers and probes are also provided. The increased sensitivity of real-time PCR for detection of S. pyogenes as well as the improved features of real-time PCR including sample containment and real-time detection of the amplified product, make feasible the implementation of this technology for routine diagnosis of S. pyogenes infections in the clinical laboratory.

The invention provides methods to detect S. pyogenes by amplifying, for example, a portion of an S. pyogenes nucleic acid derived from S. pyogenes. Nucleic acid sequences from S. pyogenes are available, e.g. in GenBank.

Primers and probes can be designed using, for example, a computer program such as OLIGO (Molecular Biology Insights, Inc., Cascade, Colo.). Important features when designing oligonucleotides to be used as amplification primers include, but are not limited to, an appropriate size amplification product to facilitate detection, similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 15 to 30 nucleotides in length. Designing oligonucleotides to be used as hybridization probes can be performed in a manner similar to the design of primers, although the members of a pair of probes preferably anneal to an amplification product. As with oligonucleotide primers, oligonucleotide probes usually have similar melting temperatures, and the length of each probe must be sufficient for sequence-specific hybridization to occur but not so long that fidelity is reduced during synthesis. Oligonucleotide probes are generally 15 to 30 nucleotides in length. Primers useful within the context of the present invention include oligonucleotides suitable in PCR reactions for the amplification of nucleic acids derived from S. pyogenes.

In describing and claiming the present invention, the terminology and definitions hereinbelow are used for the purpose of describing particular embodiments only, and are not intended to be limiting.

As used in this specification and the appended claims, the singular forms “a,” “an,” and “the” include plural referents unless the context clearly dictates otherwise.

As used herein, the term “probe” or “detection probe” refers to an oligonucleotide that forms a hybrid structure with a target sequence contained in a molecule (i.e., a “target molecule”) in a sample undergoing analysis, due to complementarity of at least one sequence in the probe with the target sequence. The nucleotides of any particular probe may be deoxyribonucleotides, ribonucleotides, and/or synthetic nucleotide analogs.

The term “primer” or “amplification primer” refers to an oligonucleotide that is capable of acting as a point of initiation for the 5′ to 3′ synthesis of a primer extension product that is complementary to a nucleic acid strand. The primer extension product is synthesized in the presence of appropriate nucleotides and an agent for polymerization such as a DNA polymerase in an appropriate buffer and at a suitable temperature.

As used herein, the term “target amplification” refers to enzyme-mediated procedures that are capable of producing billions of copies of nucleic acid target. Examples of enzyme-mediated target amplification procedures known in the art include PCR.

Within the context of the present invention, the nucleic acid “target” is a nucleic acid sequence of S. pyogenes, preferably of S. pyogenes exotoxin B (SpeB).

The most widely used target amplification procedure is PCR, first described for the amplification of DNA by Mullis et al. in U.S. Pat. No. 4,683,195 and Mullis in U.S. Pat. No. 4,683,202 and is well known to those of ordinary skill in the art. Where the starting material for the PCR reaction is RNA, complementary DNA (“cDNA”) is made from RNA via reverse transcription. A PCR used to amplify RNA products is referred to as reverse transcriptase PCR or “RT-PCR.” In the PCR technique, a sample of DNA is mixed in a solution with a molar excess of at least two oligonucleotide primers of that are prepared to be complementary to the 3′ end of each strand of the DNA duplex; a molar excess of nucleotide bases (i.e., dNTPs); and a heatstable DNA polymerase, (preferably Taq polymerase), which catalyzes the formation of DNA from the oligonucleotide primers and dNTPs. Of the primers, at least one is a forward primer that will bind in the 5′ to 3′ direction to the 3′ end of one strand of the denatured DNA analyte and another is a reverse primer that will bind in the 3′ to 5′ direction to the 5′ end of the other strand of the denatured DNA analyte. The solution is heated to 94-96° C. to denature the double-stranded DNA to single-stranded DNA. When the solution cools down and reaches the so-called annealing temperature, the primers bind to separated strands and the DNA polymerase catalyzes a new strand of analyte by joining the dNTPs to the primers. When the process is repeated and the extension products synthesized from the primers are separated from their complements, each extension product serves as a template for a complementary extension product synthesized from the other primer. As the sequence being amplified doubles after each cycle, a theoretical amplification of a huge number of copies may be attained after repeating the process for a few hours; accordingly, extremely small quantities of DNA may be amplified using PCR in a relatively short period of time.

Where the starting material for the PCR reaction is RNA, as in the case of S. pyogenes nucleic acids, complementary DNA (“cDNA”) is synthesized from RNA via reverse transcription. The resultant cDNA is then amplified using the PCR protocol described above. Reverse transcriptases are known to those of ordinary skill in the art as enzymes found in retroviruses that can synthesize complementary single strands of DNA from an mRNA sequence as a template. A PCR used to amplify RNA products is referred to as reverse transcriptase PCR or “RT-PCR.”

The terms “real-time PCR” and “real-time RT-PCR,” refer to the detection of PCR products via a fluorescent signal generated by the coupling of a fluorogenic dye molecule and a quencher moiety to the same or different oligonucleotide substrates. Examples of commonly used probes are TAQMAN® probes, Molecular Beacon probes, SCORPION® probes, and SYBR® Green probes. Briefly, TAQMAN® probes, Molecular Beacons, and SCORPION® probes each have a fluorescent reporter dye (also called a “fluor”) attached to the 5′ end of the probes and a quencher moiety coupled to the 3′ end of the probes. In the unhybridized state, the proximity of the fluor and the quencher molecules prevents the detection of fluorescent signal from the probe; during PCR, when the polymerase replicates a template on which a probe is bound, the 5′-nuclease activity of the polymerase cleaves the probe thus, increasing fluorescence with each replication cycle. SYBR Green® probes binds double-stranded DNA and upon excitation emit light; thus as PCR product accumulates, fluorescence increases. In the context of the present invention, the use of TAQMAN® probes is preferred.

The terms “complementary” and “substantially complementary” refer to base pairing between nucleotides or nucleic acids, such as, for instance, between the two strands of a double-stranded DNA molecule or between an oligonucleotide primer and a primer binding site on a single-stranded nucleic acid to be sequenced or amplified. Complementary nucleotides are, generally, A and T (or A and U), and G and C. Within the context of the present invention, it is to be understood that the specific sequence lengths listed are illustrative and not limiting and that sequences covering the same map positions, but having slightly fewer or greater numbers of bases are deemed to be equivalents of the sequences and fall within the scope of the invention, provided they will hybridize to the same positions on the target as the listed sequences. Because it is understood that nucleic acids do not require complete complementarity in order to hybridize, the probe and primer sequences disclosed herein may be modified to some extent without loss of utility as specific primers and probes. Generally, sequences having homology of 80, 90 95, 97, 98, 99% fall within the scope of the present invention. For example primer sequences or probe sequences that are 80, 90 95, 97, 98, 99% to SEQ ID Nos: 1 to 5 of the sequence listing attached hereto or of complements thereof are encompassed by the present invention. As is known in the art, hybridization of complementary and partially complementary nucleic acid sequences may be obtained by adjustment of the hybridization conditions to increase or decrease stringency, i.e., by adjustment of hybridization temperature or salt content of the buffer.

The term “hybridizing conditions” is intended to mean those conditions of time, temperature, and pH, and the necessary amounts and concentrations of reactants and reagents, sufficient to allow at least a portion of complementary sequences to anneal with each other. As is well known in the art, the time, temperature, and pH conditions required to accomplish hybridization depend on the size of the oligonucleotide probe or primer to be hybridized, the degree of complementarity between the oligonucleotide probe or primer and the target, and the presence of other materials in the hybridization reaction admixture. The actual conditions necessary for each hybridization step are well known in the art or can be determined without undue experimentation.

The term “label” as used herein refers to any atom or molecule that can be used to provide a detectable (preferably quantifiable) signal, and that can be attached to a nucleic acid or protein via a covalent bond or noncovalent interaction (e.g., through ionic or hydrogen bonding, or via immobilization, adsorption, or the like). Labels generally provide signals detectable by fluorescence, chemiluminescence, radioactivity, colorimetry, mass spectrometry, X-ray diffraction or absorption, magnetism, enzymatic activity, or the like. Examples of labels include fluorophores, chromophores, radioactive atoms, electron-dense reagents, enzymes, and ligands having specific binding partners.

As used herein, the term “sample” as used in its broadest sense to refer to any biological sample from any human or veterinary subject that may be tested for the presence or absence of one or more S. pyogenes-specific nucleic acids. The samples may include, without limitation, tissues obtained from any organ, such as for example, lung tissue; and fluids obtained from any organ such as for example, blood, plasma, serum, lymphatic fluid, synovial fluid, cerebrospinal fluid, amniotic fluid, amniotic cord blood, tears, saliva, and nasopharyngeal washes.

The term “patient” as used herein is meant to include both human and veterinary patients.

Preferred amplification primer sequences and sequences of detection probes according to the present invention are set forth in the sequence listing.

In one aspect of the invention, there is provided a method for detection of S. pyogenes in a sample comprising the steps of obtaining a biological sample from a patient; isolating nucleic acid from the sample; amplifying the nucleic acid, wherein the nucleic acid is amplified and detected with amplification primers and detection probes selected from the group depicted in the sequence listing.

In another aspect of the invention, there is provided a method for detection of S. pyogenes in a sample comprising the steps of obtaining a tissue sample from a patient; extracting nucleic acids from the sample; amplifying the nucleic acid, wherein the RNA is amplified and detected with amplification primers and detection probes as depicted in the sequence listing.

In one embodiment of the invention, the nucleic acid is selected from RNA and DNA. When the nucleic acid is RNA, it is amplified using real time RT-PCR. When the nucleic acid is DNA, it is amplified using real time PCR.

In another embodiment of the invention, the sample is a tissue fluid from a human or animal patient, which may be selected from the group consisting of blood, plasma, serum, lymphatic fluid, synovial fluid, cerebrospinal fluid, amniotic fluid, amniotic cord blood, tears, saliva, and nasopharyngeal washes.

In another embodiment of the invention, the assay is a component of a devices that is suitable in fully automated laboratories capable of extracting nucleic acids from a sample (e.g. using the epMotion System of Eppendorf International), optionally capable of reverse transcribing isolated nucleic acids, performing amplification reactions using the assay components described herein and quantitatively and qualitatively detecting nucleic acid targets, e.g. using real-time PCR.

In a further aspect, the present invention relates to a composition comprising any of the above mentioned primers and probes. Preferably, the composition comprises also ingredients, e.g. enzymes, buffers and deoxynucleotides necessary for reverse transcription and/or PCR, preferably for qualitative and/or quantitative RT-PCR. The composition may be stored in the refrigerator in a liquid state or deep-frozen in a suitable medium, or it may be lyophilized and reconstituted before use. and which may further comprises detectable probes and/or an internal control.

The present invention further provides a kit comprising the assay of the invention and optionally instructions for use.

It is to be understood that while the invention has been described in conjunction with the embodiments described herein, that the foregoing description as well as the examples that follow are intended to illustrate and not limit the scope of the invention. Other aspects, advantages and modifications within the scope of the invention will be apparent to those skilled in the art to which the invention pertains. All patents and publications mentioned herein are incorporated by reference in their entireties.

The following examples are put forth so as to provide those of ordinary skill in the art with a complete disclosure and description of how to make and use the compositions of the invention. The examples are intended as non-limiting examples of the invention. While efforts have been made to ensure accuracy with respect to variables such as amounts, temperature, etc., experimental error and deviations should be taken into account. Unless indicated otherwise, parts are parts by weight, temperature is degrees centigrade, and pressure is at or near atmospheric. All components were obtained commercially unless otherwise indicated.

### Examples

Amplification of Streptococcus pyogenes genomic DNA by specific primers and probes.

A real-time PCR assay was run with the primers depicted in SEQ ID Nos: 1 and 2 and with the probe shown in SEQ ID NO: 3, using genomic DNA of Streptococcus pyogenes Rosenbach (ATCC-12344, typing strain T1) as template. The S. pyogenes TaqMan probes are labeled with FAM reporter and BHQ-1 quencher. In addition, we have an internal extraction control using a primer/probe set specific for Lactococcus lactis subspecies Cremoris (not shown). L. lactis is added to samples during DNA extraction. A positive signal detection for L. lactis means that DNA was successfully extracted from the sample. PCR reactions were set-up either using the S. pyogenes primer/probes alone or including the L. lactis primer/probe and L. lactis genomic DNA and run on a Rotor-gene Q machine with the following cycling conditions: 95° C. for 10 mins, followed by 45 cycles of: 95° C. for 15 s and 60° C. for 60 s. Each reaction was performed in duplicate and the average CT value with standard deviation is listed in Tables 1 and 2 below.

TABLE 1

| | Ct values for S. pyogenes | | |
|---|---|---|---|
| S. pyogenes DNA | | S. pyogenes + | Ct values for |
| (ATCC VR-12344D) | S. pyogenes | L. lactis | L. lactis |
| 100pg | 23.63 ± 0.09 | 23.44 ± 0.11 | 42.23 ± 2.99 |
| 10pg | 26.81 ± 0.19 | 27.16 ± 0.05 | 34.23 ± 0.32 |
| 1pg | 30.29 ± 0.77 | 30.57 ± 0.44 | 33.26 ± 0.06 |
| 100fg | 33.87 ± 0.94 | 34.31 ± 0.36 | 32.72 ± 0.17 |
| 10fg | 36.55 ± 0.63 | 36.99 (1 replicate) | 32.43 ± 0.30 |
| 1fg | n.d. | n.d. | 32.57 ± 0.00 |

The assay with S. pyogenes alone was detected in the green channel, whereas the L. lactis signal in the duplex assay with L. lactis EC was detected in the red channel. The S. pyogenes detection will be considered positive if the Ct value is below 40. Table 2 shows the results of an experiment performed using the following copy numbers of the S. pyogenes speB plasmid instead of genomic DNA.

TABLE 2

| S. pyogenes speB | |
|---|---|
| plasmid copy number | StrepA Ct |
| 10⁸ | 13.59 ± 0.04 |
| 10⁷ | 16.84 ± 0.00 |
| 10⁶ | 20.28 ± 0.06 |
| 10⁵ | 23.64 ± 0.11 |
| 10⁴ | 26.66 ± 0.26 |
| 10³ | 30.23 ± 0.60 |
| 10² | 33.02 ± 0.30 |
| 10 | 37.32 (1 out of 2) |
| 5 | 40.04 (1 out of 2) |
| 2.5 | n.d. |

TABLE 3
Another inventive assay was run:

| Parameter | FRD Requirements | Test Results |
|---|---|---|
| Analytical | ≦500 CFU/mL (≧95%) | 2.3 copies/μL or 250 |
| Sensitivity | | CFU/mL (96.7%) Dynamic |
| (LoD) | | range: 2.7-2 × 10⁵ copies/μl |
| Analytical | To detect ≧95% of | Able to detect 9 strains of |
| Reactivity | known S.pyogenes strains | S.pyogenes from ATCC |
| | with homologous se- | tested |
| | quence tested | |
| Analytical | No cross-reactivity with | No cross-reactivity with 25 |
| Specificity | other clinical | microorganisms tested |
| | microorganisms tested | |
| Clinical | ≧95% | 99.6% |
| Specificity | | |
| Clinical | ≧95% | 99.9% |
| Sensitivity | | |
| Reproducibility | ≧99% | ≧99% |
| Turn Around | 4 hours including sample | 2 hrs 57 mins |
| Time | lysis and extraction | |
| Temperature | Assay performed at room | Stable for 3 hours at room |
| | temperature | temperature |
| Specimen Types | Wound and throat swab | Wound and throat swab |
| Pretreatment | Not required | No pre-treatment required |
| Storage | Assay kit to be stored | −20 ± 5° C. |
| conditions | at −20 ± 5° C. | |
| Minimum ex- | 9 months from the date | Stable for at least 2 years |
| piration date | of manufacturing | at −20° C. |

As shown above, the assay of the invention is suitable for rapid, surprisingly sensitive and specific diagnosis of small amounts of nucleic acids of S. pyogenes in a clinical sample.

This assay may also be designed as a duplex assay. One advantage of this is it allows L. lactis to be added to clinical samples. L. lactis DNA may serve as an internal extraction control to show that DNA is successfully extracted. Another option is the use of a plasmid comprising a sequence that is used as extraction control. Such a sequence is shown e.g. in SEQ ID No: 7. If the real-time PCR reaction yields negative signals for both S. pyogenes and L. lactis, DNA extraction was unsuccessful. This control eliminates a false negative reading. Further, as shown in the experimental data, the presence of L. lactis DNA and L. lactis primer/probes does not affect the sensitivity of the detection, which was 10 fg of total S. pyogenes DNA. Further sensitivity studies using a fragment of the SpeB gene cloned into a plasmid, with 10-fold dilutions of the plasmid have shown that down to 5 copies of the gene can be detected.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for the detection of the presence or absence of at least one nucleic acid specific for S. pyogenes in a biological sample, wherein the method comprises:
(a) isolating nucleic acids from the biological sample and optionally performing a reverse transcription step, and
(b) conducting real-time PCR, wherein a primer set that is specific for S. pyogenes-specific nucleic acids is used, wherein the S. pyogenes-specific primer set comprises oligonucleotide sequences set forth in SEQ ID NOs: 1 and 2 or SEQ ID NOs: 4 and 5, complements thereof or sequences having at least 80, 85, 90, or 95% identity to any of SEQ ID NOs: 1, 2, 4, and 5 or complements thereof, and wherein further at least one probe specifically binding to a nucleic acid of S. pyogenes is used, wherein the at least one probe comprises the oligonucleotide set forth in SEQ ID NO: 3 or a complement thereof, or a sequence having at least 80, 85, 90, or 95% identity to SEQ ID NO: 3 or a complement thereof.

2. The method according to claim 1, wherein the primers and/or probe carry/carries a fluorescent moiety.

3. An in vitro method for the diagnosis of an S. pyogenes infection in a subject comprising performing the method steps according to claim 1.

4. A method for monitoring the treatment of S. pyogenes infection, said method comprising performing the method according to claim 3 before treatment with at least one anti-bacterial drug and/or during and/or after treatment with said drug.

5. A real-time PCR assay for simultaneous detection of at least one nucleic acid specific for S. pyogenes in a biological sample as defined in claim 1.

6. The assay according to claim 5, wherein the assay is adapted for use in a fully automated laboratory.

7. A composition comprising primers and/or probes having oligonucleotide sequences as set forth in claim 1.

8. A kit for the detection of S. pyogenes in a biological sample comprising primers and/or probes having oligonucleotide sequences as set forth in claim 1, further comprising instructions for use.

9. The kit according to claim 8, wherein said kit further comprises enzymes, deoxynucleotides, and/or buffers for performing a reverse transcription step and/or a PCR step.

10. The kit according to claim 8 further comprising reagents for the isolation of nucleic acids from a biological sample.


SEQUENCE LISTING (7 sequences)

SEQ ID NO: 1 - DNA, 18 bp, Streptococcus pyogenes
ggcttcttccgtcttgac

SEQ ID NO: 2 - DNA, 20 bp, Streptococcus pyogenes
cctacaacagcactttggta

SEQ ID NO: 3 - DNA, 21 bp, Streptococcus pyogenes
cgccgccaccagtaccaagag

SEQ ID NO: 4 - DNA, 20 bp, Streptococcus pyogenes
tgacggacgtaacttctacc

SEQ ID NO: 5 - DNA, 19 bp, Streptococcus pyogenes
gcctacaacagcactttgg

SEQ ID NO: 6 - DNA, 527 bp, Artificial sequence
  Plasmid comprised positive control sequence
gcggccgcctggggttggggtggagtctctgacggcttcttccgtcttgacgcactaaacccttcagctcttggtactggtggcggcgcaggcggcttcaacggttaccaaagtgctgttgtaggcatcaaaccttagtatggaaatgcatggtggtcttgacgacgggctcgtagaccgtgcggatcttgccctccggccagtccgacggattggtcacccggatttcggtgccgtcgtctttgtgcacccgatacaccacattgggtgaggtcgagatcaggtccaggccgaactcgcgctcaaggcgctcacgggtgatctccatgtgcagcaggcccaagaaaccgcaccggaacccgccagcagggctcttcgtcagctcccactcgtagccgtacaggatctcgaggaaactgttgtcccatttcgtcggggtgttcgtccatacgacctcgatgccggtggtgatcgcgtccttaccggttccggtgccatacgagctcttccagcccaagcccatctgc

SEQ ID NO: 7 - DNA, 443 bp, Artificial sequence
  Plasmid comprised sequence used as extraction control
gctaatacgactcactatagggagatagcatttaggtgacactatagaagttagtacctgggaagcaagccgcggaaatgatcagaagacgtgcgaattcctcagggattattgtggccacgaaggataacgttaaaaccgttgattctttcatgatgaattttgggaaaagcacacgctgtcagttcaagaggttattcattgatgaagggttgatgttgcatactggttgtgttaattttcttgtggcgatgtcattgtggtaagatttcacgtcctgccttaggtattcgtatggttgacctatctcaattatcaacaaatgatagttctcaactgaaattacctagcagcgtaactggtggagtggttgtctactctgttcaagcgggtcttcctgctgccacagctggaaaaaaaaaaaaaaaaaaaaaaaaaaaaaa